(19) United States
(12) Patent Application Publication
(10) Pub. No.: US 2026/0074024 A1
(43) Pub. Date: Mar. 12, 2026

(54) INFORMATION PROCESSING METHOD, INFORMATION PROCESSING SYSTEM, AND PROGRAM

(71) Applicant: Panasonic Intellectual Property Management Co., Ltd., Osaka (JP)

(72) Inventors: TOMOYASU YOKOYAMA, Osaka (JP); KAZUHIDE ICHIKAWA, Kyoto (JP)

(21) Appl. No.: 19/389,522

(22) Filed: Nov. 14, 2025

Related U.S. Application Data
(63) Continuation of application No. PCT/JP2024/019018, filed on May 23, 2024.

(30) Foreign Application Priority Data
Jun. 1, 2023 (JP) 2023-091015

(51) Int. Cl. G16C 20/50; G16C 20/80

(52) U.S. Cl. CPC G16C 20/50; G16C 20/80

(57) ABSTRACT

An information processing method performed by a computer includes acquiring a phase diagram which includes a target compound made of four or more kinds of elements and in which each of two or three kinds of substances is placed on an apex to be two-dimensionally represented and outputting the acquired phase diagram. At least one of the two or three kinds of substances is a compound made of at least two kinds of elements, among the four or more kinds of elements.

### BACKGROUND

### 1. Technical Field

The present disclosure relates to a technique for displaying a phase diagram of a multi-component compound.

### 2. Description of the Related Art

A phase diagram is a “map” for material search. A user can easily understand the synthesis conditions (composition, raw materials, temperature, pressure, and/or the like) required to synthesize target materials by referring to the phase diagram. In addition, even in a case of a material for which no experimental report has been made, predicting the phase diagram using first-principles calculation, a machine learning-based prediction model, or the like may lead to the detection of a new high-functional material.

Japanese Unexamined Patent Application Publication No. 2006-125952 discloses a surface analysis apparatus that performs phase analysis using the phase diagram.

### SUMMARY

One non-limiting and exemplary embodiment provides an information processing method capable of outputting a phase diagram that is easily visually recognized by users for a multi-component compound.

In one general aspect, the techniques disclosed here feature an information processing method performed by a computer. The information processing method includes acquiring a phase diagram which includes a target compound made of four or more kinds of elements and in which each of two or three kinds of substances is placed on an apex to be two-dimensionally represented and outputting the acquired phase diagram. At least one of the two or three kinds of substances is a compound made of at least two kinds of elements, among the four or more kinds of elements. It should be noted that general or specific embodiments may be implemented as an apparatus, a system, an integrated circuit, a computer program, a computer-readable storage medium, or any selective combination of the method, the apparatus, the system, the integrated circuit, the computer program, and the computer-readable recording medium. The computer-readable recording medium includes a non-volatile recording medium, such as a compact disk read only memory (CD-ROM).

According to the present disclosure, it is possible to output the phase diagram that is easily visually recognized by users for the multi-component compound.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example of a phase diagram of a ternary system made of three kinds of elements;

FIG. 2 is a diagram illustrating an example of the phase diagram of a quaternary system made of four kinds of elements;

FIG. 3 is a diagram illustrating an example of a phase diagram before dimensionality reduction and a phase diagram after the dimensionality reduction;

FIG. 4 is a diagram illustrating another example of a phase diagram before the dimensionality reduction and a phase diagram after the dimensionality reduction;

FIG. 5 is a block diagram illustrating the entire configuration including an information processing system according to a first embodiment;

FIG. 6 is a diagram illustrating examples of images displayed in a display in the first embodiment;

FIG. 7 is a diagram illustrating examples of a second image displayed in the display in the first embodiment;

FIG. 8 is a diagram illustrating an example of a third image displayed in the display in the first embodiment;

FIG. 9 is a flowchart illustrating an example of the operation of the information processing system according to the first embodiment;

FIG. 10 is a table indicating an example of second information;

FIG. 11 is a sequence diagram illustrating an example of the entire operation including the information processing system according to the first embodiment;

FIG. 12 is a diagram illustrating examples of images displayed in the display in a second embodiment;

FIG. 13 is a flowchart illustrating an example of the operation of an information processing system according to the second embodiment;

FIG. 14 is a sequence diagram illustrating an example of the entire operation including the information processing system according to the second embodiment;

FIG. 15 is a diagram illustrating an example of the second image displayed in the display in a third embodiment;

FIG. 16 is a flowchart illustrating an example of the operation of an information processing system according to the third embodiment;

FIG. 17 is a sequence diagram illustrating an example of the entire operation including the information processing system according to the third embodiment;

FIG. 18 is a diagram illustrating an example of the second image displayed in the display in a fourth embodiment; and

FIG. 19 is a diagram illustrating an example of a phase diagram subjected to the dimensionality reduction when a target compound is a quinary system.

### DETAILED DESCRIPTIONS

### Underlying Knowledge Forming Basis of the Present Disclosure

In search for a new material, a phase diagram takes a role of a “map”. Here, the phase diagram is a diagram in which multiple substances (elements (atoms) or compounds) composing a material are represented at apexes and one or more compounds made of the multiple substances are represented on lines. The composition of the material and the composition representing the phase thermodynamically coexisting with the material are displayed in the phase diagram. In addition, in the phase diagram, thermodynamic convex hull energy is zero, that is, the compositions representing thermodynamically stable phases are connected with each other using straight lines.

Since a user can easily understand synthesis conditions (composition, raw materials, temperature, pressure, and/or the like) required to synthesize target materials by referring to the phase diagram, the phase diagram is essential for material search. In addition, even in a case of a material for which no experimental report has been made, predicting the phase diagram using first-principles calculation, a machine learning-based prediction model, or the like may lead to the detection of a new high-functional material.

Wide-range material search has been enabled in recent years due to development of Materials Informatics. For example, as typified by high entropy alloy, research and development of a multi-component compound made of four or more kinds of elements, which were previously difficult to research and develop, has been actively advanced.

Although the search for the multi-component compound has been enabled, as described above, there is a problem in that it is difficult to design a material from the phase diagram in the related art due to the diversification of the target materials. This problem will be described with reference to the drawings.

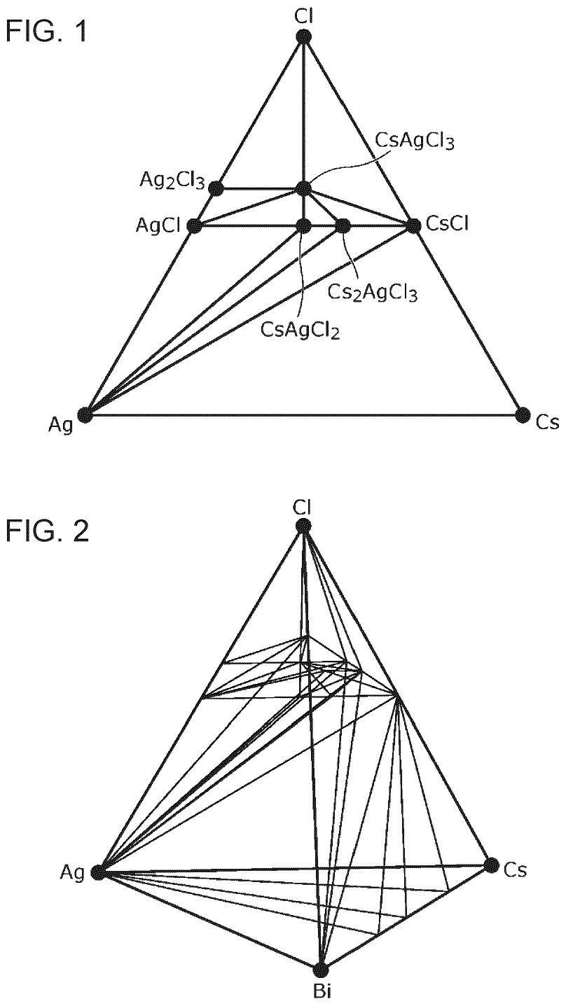

FIG. 1 is a diagram illustrating an example of the phase diagram of a ternary system made of three kinds of elements. In the example illustrated in FIG. 1, the three kinds of elements are silver (Ag), chlorine (CI), and cesium (Cs). FIG. 2 is a diagram illustrating an example of the phase diagram of a quaternary system made of four kinds of elements. In the example illustrated in FIG. 2, the four kinds of elements are silver, chlorine, cesium, and bismuth (Bi). Since the phase diagram of the ternary system made of the three elements is represented as a regular triangle on a two-dimensional plane, as illustrated in FIG. 1, users easily visually recognize the phase diagram of the ternary system. The phase diagram represented on the two-dimensional plane, as the one illustrated in FIG. 1, has been used in the material search from long ago and is particularly easily visually recognized by the user performing the material search. In contrast, since the phase diagram of the quaternary system made of the four elements is represented as a regular tetrahedron on a three-dimensional space, as illustrated in FIG. 2, it is difficult for users to visually recognize the phase diagram of the quaternary system, compared to two-dimensional representations. In addition, since a phase diagram of a quinary system made of five elements is represented as a regular pentatope on a four-dimensional space, it is difficult for users to visually recognize the phase diagram of the quinary system. Since it is difficult for users to visually recognize the phase diagrams in the related art due to such diversification of the materials, this restricts design and development of the multi-component compounds.

In order to resolve the above problems, an information processing method according to a first aspect of the present disclosure, which is performed by a computer, includes acquiring a phase diagram which includes a target compound made of four or more kinds of elements and in which each of two or three kinds of substances is placed on an apex to be two-dimensionally represented and outputting the acquired phase diagram. At least one of the two or three kinds of substances is a compound made of at least two kinds of elements, among the four or more kinds of elements.

With this information processing method, it is possible to output the phase diagram that is easily visually recognized by users for the multi-component compound.

For example, the information processing method according to a second aspect of the present disclosure may further include acquiring a multi-component phase diagram which includes the target compound and in which each of the four or more kinds of elements is placed on an apex to be at least three-dimensionally represented, in the first aspect. In the first aspect, in extracting the phase diagram, at least one phase diagram may be extracted from the acquired multi-component phase diagram.

With this information processing method, it is possible to output the phase diagram that is easily visually recognized by users for the multi-component compound.

For example, in the information processing method according to a third aspect of the present disclosure, in the second aspect, the multi-component phase diagram may include multiple compounds formed by combining two or more kinds of elements, among the four or more kinds of elements. In the second aspect, in extracting the phase diagram, the phase diagram in which at least one compound, among the multiple compounds, is placed at a position other than the apex may be extracted.

With this information processing method, it is possible to output the phase diagram that is easily visually recognized by users for the multi-component compound.

An information processing system according to a fourth aspect of the present disclosure includes a display controller that, after a first image accepting input of first information concerning a target compound made of four or more kinds of elements is displayed in a display, displays a second image indicating a phase diagram that is generated based on the input first information and that is two-dimensionally represented in the display. The phase diagram includes three or more apexes, and the target compound and multiple compounds each made of at least two kinds of elements, among the four or more kinds of elements, are placed in the phase diagram. At least one compound, among the multiple compounds, is placed on at least one apex, among the three or more apexes in the phase diagram.

With this information processing system, it is possible to output the phase diagram that is easily visually recognized by users for the multi-component compound.

For example, in the information processing system according to a fifth aspect of the present disclosure, in the fourth aspect, the phase diagram may include one or more different phase diagrams, and the display controller may display the second image indicating the one or more phase diagrams in the display.

With this information processing system, it is possible to output the phase diagram that is easily visually recognized by users for the multi-component compound.

For example, in the information processing system according to a sixth aspect of the present disclosure, in the fifth aspect, the one or more phase diagrams may be arranged in the descending order of the numbers of the compounds included in the phase diagrams in the second image.

With this information processing system, it is possible to output the phase diagram that is easily visually recognized by users for the multi-component compound.

For example, in the information processing system according to a seventh aspect of the present disclosure, in the fifth or sixth aspect, upon acceptance of selection of any one phase diagram from the one or more phase diagrams, the display controller may display the second image indicating the selected one phase diagram in the display.

With this information processing system, it is possible to output the phase diagram that is easily visually recognized by users for the multi-component compound.

For example, in the information processing system according to an eighth aspect of the present disclosure, in any one of the fourth to seventh aspects, the display controller may display the second image further including a multi-component phase diagram that is at least three-dimensionally represented in the display. In any one of the fourth to seventh aspects, each of the four or more kinds of elements may be placed on an apex and the target compound may be placed in the multi-component phase diagram.

With this information processing system, it is possible to output the phase diagram that is easily visually recognized by users for the multi-component compound.

For example, in the information processing system according to a ninth aspect of the present disclosure, in the eighth aspect, the display controller may display the second image in which a plane or a line representing at least one phase diagram is superimposed on the multi-component phase diagram in the display.

With this information processing system, it is possible to output the phase diagram that is easily visually recognized by users for the multi-component compound.

For example, in the information processing system according to a tenth aspect of the present disclosure, in any one of the fourth to ninth aspects, the one or more phase diagrams may be each represented in a triangle and may be differentiated in at least one of the shape of the triangle, the substance corresponding to each apex of the triangle, or the area of the triangle.

With this information processing system, it is possible to output the phase diagram that is easily visually recognized by users for the multi-component compound.

For example, in the information processing system according to an eleventh aspect of the present disclosure, in any one of the fourth to tenth aspects, the display controller may display the first image accepting input of target elements included in the target compound and an image for accepting input of material properties of the target compound in the display as the first information, and
the display controller may display an image including one or more target compounds that are predicted based on the input target elements and the material properties and that include the target elements and the material property value of the one or more target compounds in the display.

With this information processing system, by specifying multiple elements to be included in the multi-component compound even without specification of a desired multi-component compound by the user, it is possible to output the phase diagram that is easily visually recognized by users for the multi-component compound made of the specified multiple elements.

For example, in the information processing system according to a twelfth aspect of the present disclosure, in the eleventh aspect, upon acceptance of selection of any one target compound from the one or more target compounds, the display controller may display the second image including the selected one target compound and an image representing the material property value of the one target compound in the display.

With this information processing system, it is possible to output the phase diagram that is easily visually recognized by users for the material properties of the multi-component compound.

For example, in the information processing system according to a thirteenth aspect of the present disclosure, in any one of the fourth to twelfth aspects, the display controller may display the second image representing the phase diagram that is generated based on at least one of a temperature condition or a pressure condition of the target compound in the display.

With this information processing system, it is possible to output the phase diagram that is easily visually recognized by users for the multi-component compound in consideration of at least one of the temperature or the pressure.

For example, in the information processing system according to a fourteenth aspect of the present disclosure, in any one of the fourth to thirteenth aspects, the display controller may display the second image representing the phase diagram that further includes an energy axis indicating the energy of each of multiple substances included in the phase diagram in the display.

With this information processing system, it is possible to output the phase diagram that is easily visually recognized by users for the stability of the compounds included in the multi-component compound.

A program according to a fifteenth aspect of the present disclosure causes a computer to perform acquiring a phase diagram which includes a target compound made of four or more kinds of elements and in which each of two or three kinds of substances is placed on an apex to be two-dimensionally represented and outputting the acquired phase diagram. At least one of the two or three kinds of substances is a compound made of at least two kinds of elements, among the four or more kinds of elements.

With this program, it is possible to output the phase diagram that is easily visually recognized by users for the multi-component compound.

Characteristic processes included in the information processing method of the present disclosure may be realized as a computer program executed by a computer. The computer program may be distributed via a non-transitory computer-readable recording medium, such as a compact disk read only memory (CD-ROM), or a communication network, such as the Internet.

In other words, according to the technique of the present disclosure, reducing the order of the phase diagram of the multi-component compound made of the four or more kinds of elements to the two-dimensionally represented phase diagram enables users referring to the phase diagram to easily understand the phase diagram. However, it is necessary to perform the reduction in the order of the phase diagram of the multi-component compound so that information necessary for material design is included in the phase diagram the order of which is reduced. The information necessary for the material design corresponds to phases coexisting with a target phase and phases, which is raw materials, in the phase diagram.

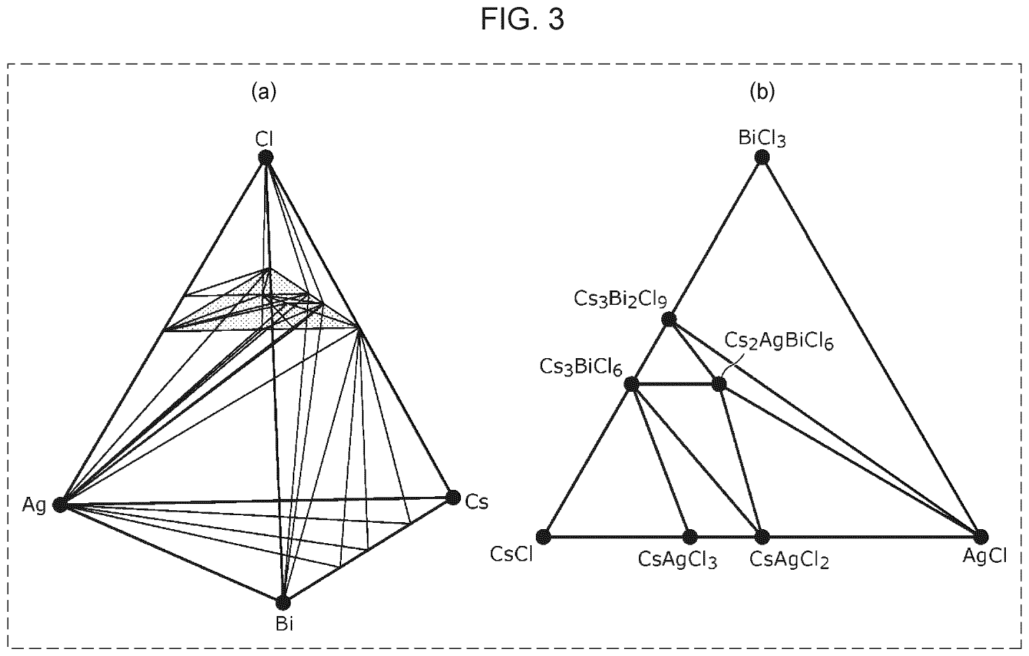

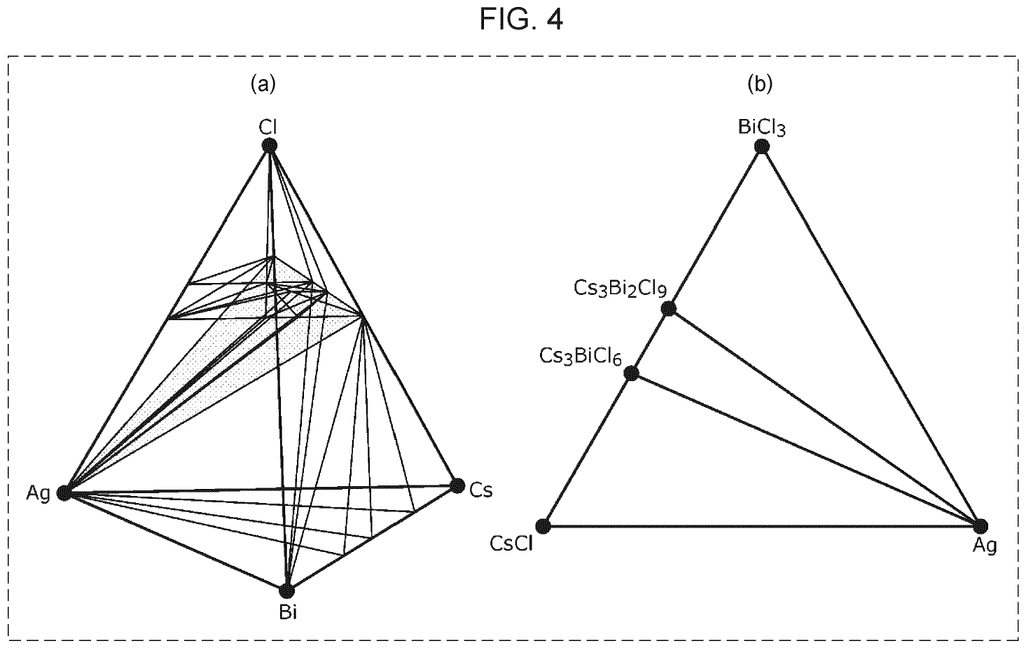

For example, a case will now be considered, in which a quaternary system made of four kinds of elements (Cs, Ag, Bi, and Cl), which is Cs₂AgBiCl₆, is the target material. FIG. 3 is a diagram illustrating an example of a phase diagram before the dimensionality reduction and a phase diagram after the dimensionality reduction. FIG. 3(a) illustrates the phase diagram before the dimensionality reduction, which is represented in a three-dimensional space having Cs, Ag, Bi, and Cl on the respective apexes. FIG. 3(b) results from cutting out the hatched plane in FIG. 3(a) and illustrates the phase diagram after the dimensionality reduction, which is represented in a two-dimensional plane having CsCl, AgCl, and BiCl₃ on the respective apexes. FIG. 4 is a diagram illustrating another example of a phase diagram before the dimensionality reduction and a phase diagram after the dimensionality reduction. FIG. 4(a) illustrates the phase diagram before the dimensionality reduction, which is represented in a three-dimensional space having Cs, Ag, Bi, and Cl on the respective apexes. FIG. 4(b) results from cutting out the hatched plane in FIG. 4(a) and illustrates the phase diagram after the dimensionality reduction, which is represented in a two-dimensional plane having CsCl, Ag, and BiCl₃ on the respective apexes.

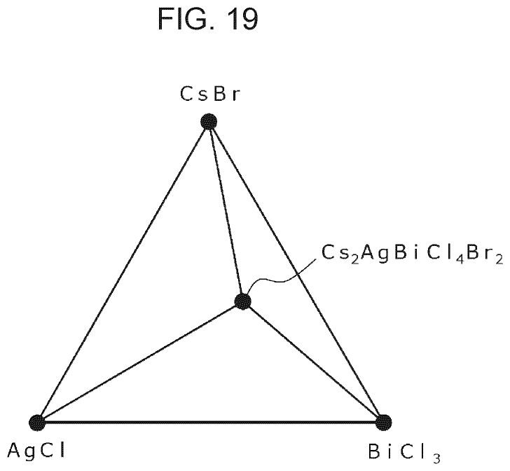

FIG. 19 is a diagram illustrating an example of a phase diagram subjected to the dimensionality reduction when the target compound is a quinary system. FIG. 19 illustrates the phase diagram after the dimensionality reduction, which is represented in a two-dimensional plane having CsBr, AgCl, and BiCl₃ on the respective apexes.

In the phase diagram before the dimensionality reduction (refer to FIG. 3(a) or FIG. 4(a)), the phases coexisting with Cs₂AgBiCl₆, which is the target material, are Cs₃Bi₂Cl₉, Cs₃BiCl₆, CsAgCl₂, CsAgCl₃, and AgCl. All the information about Cs₃Bi₂Cl₉, Cs₃BiCl₆, CsAgCl₂, CsAgCl₃, and AgCl, which are the phases coexisting with Cs₂AgBiCl₆, are extracted in the phase diagram after the dimensionality reduction in FIG. 3(b). Accordingly, the user can visually understand which phases Cs₂AgBiCl₆, which is the target material, coexists with by referring to the phase diagram after the dimensionality reduction in FIG. 3(b) and can easily perform the material design.

In contrast, in the phase diagram after the dimensionality reduction in FIG. 4(b), the information about Cs₂AgBiCl₆, which is the target material, is missing. Accordingly, the user cannot visually understand which phases Cs₂AgBiCl₆, which is the target material, coexists with even by referring to the phase diagram after the dimensionality reduction in FIG. 4(b) and may have difficulty performing the material design.

Accordingly, in the dimensionality reduction of the phase diagram of the multi-component compound, reducing the order of the phase diagram so that the phase diagram is two-dimensionally represented so as to include the target compound and the phases coexisting with the target compound or the phases, which is the raw materials, enables the user to easily understand the phase diagram of the multi-component compound.

Embodiments will herein be described specifically with reference to the drawings.

All the respective embodiments described below indicate comprehensive or specific examples of the present disclosure. Numerical values, shapes, materials, components, the positions where the components are arranged, the connection mode of the components, steps, and the order of the steps, which are indicated in the respective embodiments described below, are only examples and are not intended to limit the present disclosure. Among the components in the respective embodiments described below, the components that are not described in the independent claims indicating the highest order concepts are described as arbitrary components. The respective diagrams are schematic diagrams and are not necessarily strictly illustrated. The same reference numerals are added to the same components in the respective drawings.

In the information processing system according to each embodiment of the present disclosure, all the components may be included in one computer. Alternatively, the information processing system according to each embodiment of the present disclosure may be composed of a system in which the multiple components are distributed to multiple computers.

### First Embodiment

An information processing system (an information processing method or a program) according to a first embodiment of the present disclosure will be described in detail with reference to the drawings.

### Information Processing System

The configuration of the information processing system used in the first embodiment will now be described.

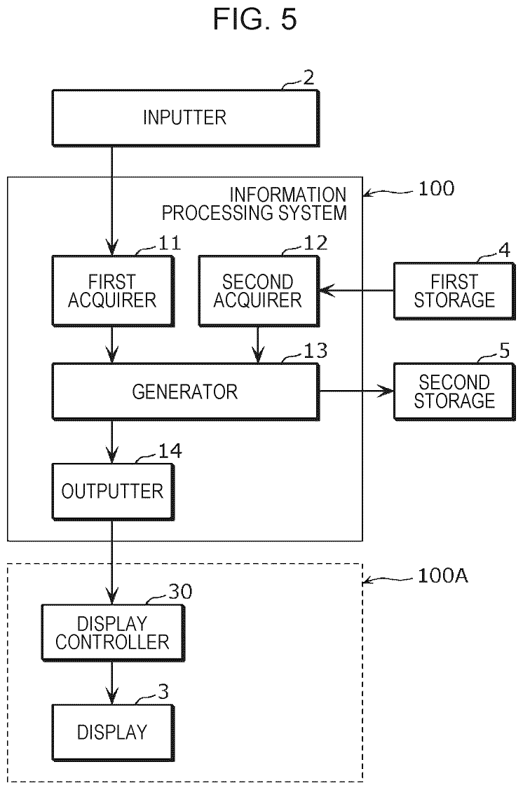

FIG. 5 is a block diagram illustrating the entire configuration including an information processing system 100 according to the first embodiment. The information processing system 100 is composed of, for example, a personal computer or a computer, such as a server. In other words, the information processing system 100 may be realized by, for example, cloud computing. The information processing system 100 is described as a stationary computer in the first embodiment.

The information processing system 100 includes a first acquirer 11, a second acquirer 12, a generator 13, and an outputter 14. In addition, an inputter 2, a display controller 30, a display 3, a first storage 4, and a second storage 5 are connected to the information processing system 100. Each of the inputter 2, the display controller 30, and the display 3 is composed of, for example, an information terminal, such as a smartphone, a tablet terminal, or a personal computer, used by a user.

Each of the inputter 2, the display controller 30, the first storage 4, and the second storage 5 may be connected to the information processing system 100 via a local area network (LAN) or the like or may be connected to the information processing system 100 via a network, such as the Internet.

The inputter 2 is an input interface accepting input by the user and is composed of, for example, a keyboard, a touch sensor, a touch pad, a mouse, or the like. The inputter 2 accepts an input operation by the user and outputs a signal corresponding to the input operation to the information processing system 100. Although the display 3 and the inputter 2 are independently configured in the present disclosure, the display 3 and the inputter 2 may be integrally configured as, for example, a touch panel. Although the information processing system 100 does not include the display 3 and the inputter 2 in the present disclosure, the information processing system 100 may include the display 3 and the inputter 2.

The inputter 2 accepts input of first information concerning a target compound made of four or more kinds of elements. The target compound may be, for example, a compound desired by the user or may simply be a compound to be output. The first information is, for example, composition information indicating the composition of the target compound. The first information may be, for example, element information indicating the elements included in the target compound, atomic ratio information indicating the atomic ratios of the multiple elements included in the target compound, crystal structure information indicating the crystal structure of the target compound, or the like, instead of the composition information. The first information is the composition information in the first embodiment.

The display controller 30 displays an image or the like in the display 3 based on information output from the outputter 14 in the information processing system 100.

The display 3 displays the image or the like under the control of the display controller 30. Although the display 3 is, for example, a liquid crystal display, a plasma display, an organic electro-luminescence (EL) display, or the like, the display 3 is not limited to these.

The first storage 4 is a recording medium for storing a phase diagram database. The recording medium is, for example, a hard disk drive, a random access memory (RAM), a read only memory (ROM), a semiconductor memory, or the like. Such a recording medium may be a volatile recording medium or a non-volatile recording medium.

The phase diagram database includes compound data concerning the compound. The compound data included in the phase diagram database is exemplified by, for example, thermodynamic data about an individual element (for example, Cs, Ag, Bi, Cl, or the like) and thermodynamic data about a compound (for example, CsCl, AgCl, BiCl₃, Cs₂AgBiCl₆, or the like). Here, the thermodynamic data is free energy at an arbitrary temperature and at an arbitrary pressure of the element or the compound. The thermodynamic data included in the phase diagram database may include data that is experimentally measured or may include data that is predicted through simulation, such as the first-principles calculation. The compound data may include material property information indicating the material property values, such as structure information, energy information, band gap, volume, ionic conductivity, and/or dielectric constant, of the compound. The structure information may include, for example, the composition of the compound, the crystal system, the space group, the lattice constant, the atomic coordinate, or the like. The energy information may include, for example, thermodynamic convex hull energy, or formation energy.

The second storage 5 is a recording medium for storing third information indicating the phase diagram selected by the user, among one or more phase diagrams generated by the generator 13. The recording medium is, for example, a hard disk drive, a RAM, a ROM, a semiconductor memory, or the like. Such a recording medium may be a volatile recording medium or a non-volatile recording medium.

The first acquirer 11 acquires the first information concerning the target compound, such as Cs₂AgBiCl₆. The first acquirer 11 is a component configured to acquire the first information in the information processing method of the present disclosure. Specifically, the first acquirer 11 acquires the first information input by the user with the inputter 2. As described below, the user performs an operation to input the first information while looking at a first image accepting input of the first information, which is displayed in the display 3.

The second acquirer 12 acquires second information concerning the composition of the target compound from the first storage 4. The second acquirer 12 is a component configured to acquire the second information in the information processing method of the present disclosure. Specifically, the second acquirer 12 reads out data concerning the composition of the target compound, indicated by the first information, from the first storage 4 based on the first information input by the user with the inputter 2 to acquire the read-out data as the second information. The data concerning the composition of the target compound may include data concerning the four or more kinds of elements composing the target compound and data concerning the compound formed by combining two or more kinds of elements, among the four or more kinds of element.

The generator 13 generates the third information indicating the one or more phase diagrams based on the first information acquired by the first acquirer 11 and the second information acquired by the second acquirer 12. The generator 13 is a component configured to generate the third information in the information processing method of the present disclosure. Each of the one or more phase diagrams is a diagram which includes the target compound and in which each of two or three kinds of substances (elements or compounds) is placed on an apex to be two-dimensionally represented. At least one of the two or three kinds of substances is the compound made of at least two kinds of elements, among the four or more kinds of elements composing the target compound.

In the first embodiment, the generator 13 generates (acquires) a multi-component phase diagram indicating the target compound based on the first information and the second information. The multi-component phase diagram is a diagram which includes the target compound and in which each of the four or more kinds of elements composing the target compound is placed on an apex to be at least three-dimensionally represented. The generator 13 extracts at least one phase diagram from the generated (acquired) multi-component phase diagram. In particular, in the first embodiment, the generator 13 extracts the phase diagram in which at least one compound, among the multiple compounds in the multi-component phase diagram, is placed at a position other than the apex. The process performed by the generator 13 will be described in detail below.

The outputter 14 outputs an image or the like to the display controller 30 to cause the display controller 30 to display the image or the like in the display 3. The outputter 14 outputs the third information generated by the generator 13. The outputter 14 is a component configured to output the third information in the information processing method of the present disclosure. Specifically, the outputter 14 causes the display controller 30 to display a second image representing the third information generated by the generator 13 in the display 3 to output the third information. As described below, the user performs an operation to select fourth information to be stored in the second storage 5 while looking at the second image displayed in the display 3.

### Examples of Use

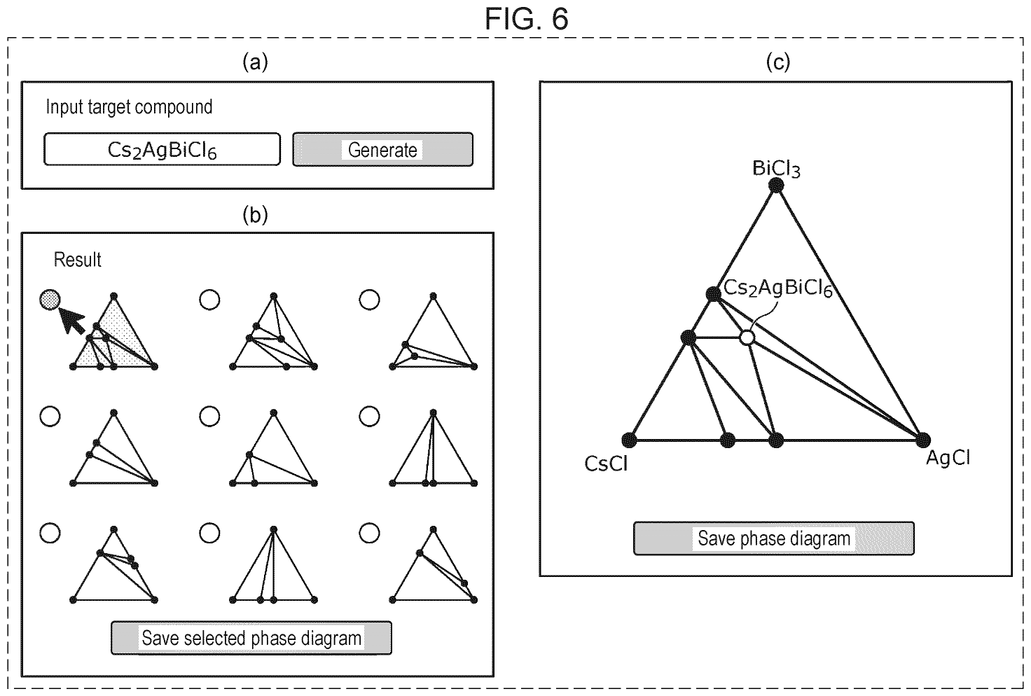

Examples of use of the information processing system 100 according to the first embodiment will now be described. FIG. 6 illustrates images displayed in the display 3 in the first embodiment. FIG. 6(a) illustrates an example of the first image displayed in the display 3. The first image is an image accepting input of the first information. In the first embodiment, the first image includes a first information input field used to input the first information concerning the target compound, such as the composition information (for example, Cs₂AgBiCl₆) about the target compound, and an execution icon “Generate”.

A text box used to specify the composition of the target compound is displayed in the first information input field. The user inputs the composition of a desired target compound in the text box and selects the execution icon. As a result, the first acquirer 11 acquires the composition information about the target compound as the first information. In this case, the second acquirer 12 acquires the data concerning the composition of the target compound, which is input by the user, from the first storage 4 as the second information. The generator 13 generates the third information indicating the one or more phase diagrams based on the first information and the second information.

The first image may include an input field used to specify the number of the phase diagrams to be displayed in the second image described below. Upon input of specification of the number of the phase diagrams in the input field by the user, a list of the phase diagrams of the specified number is displayed in the second image.

The first image may include an input field used to specify which database is used to generate the third information when multiple databases exist as the first storage 4. Upon input of specification of the database in the input field by the user, the generator 13 generates the third information using the information stored in the database. The multiple databases may include, for example, a database storing the second information that is experimentally acquired, a database storing the second information acquired through calculation, or the like. The database storing the second information acquired through calculation may include, for example, databases the calculation methods of which are different from each other, such as a database storing the second information acquired through the first-principles calculation and a database storing the second information acquired through machine learning.

FIG. 6(b) illustrates an example of the second image displayed in the display 3. The second image is displayed in the display 3 after the user selects the execution icon in the first image and the generator 13 generates the third information. The second image includes a list of one or more (nine here) phase diagrams generated by the generator 13, a selection button provided for each of the one or more phase diagrams, and an execution icon “Save selected phase diagram”. The user can confirm all the compounds coexisting with the target compound on the two-dimensional plane by referring to the multiple phase diagrams displayed in the display 3. When the user has an interested compound, the user can search for the phase diagram corresponding to the compound from the multiple phase diagrams to understand the relationship with the target compound.

For example, upon selection of any phase diagram by the user, the second image including an image resulting from enlargement of the selected phase diagram and an execution icon “Save phase diagram”, is displayed in the display 3, as illustrated in FIG. 6(c). A white circle in the phase diagram represents the target compound and black circles in the phase diagram represent the elements or the compounds. The user selects the execution icon “Save phase diagram” on the second image illustrated in FIG. 6(c). Alternatively, the user selects the execution icon “Save selected phase diagram” after selecting any button on the second image illustrated in FIG. 6(b). Then, the fourth information indicating the phase diagram selected by the user is saved in the second storage 5. The fourth information may be saved in the second storage 5 as image data about the phase diagram or may be saved in the second storage 5 as table data in which the pieces of information included in the phase diagram are listed.

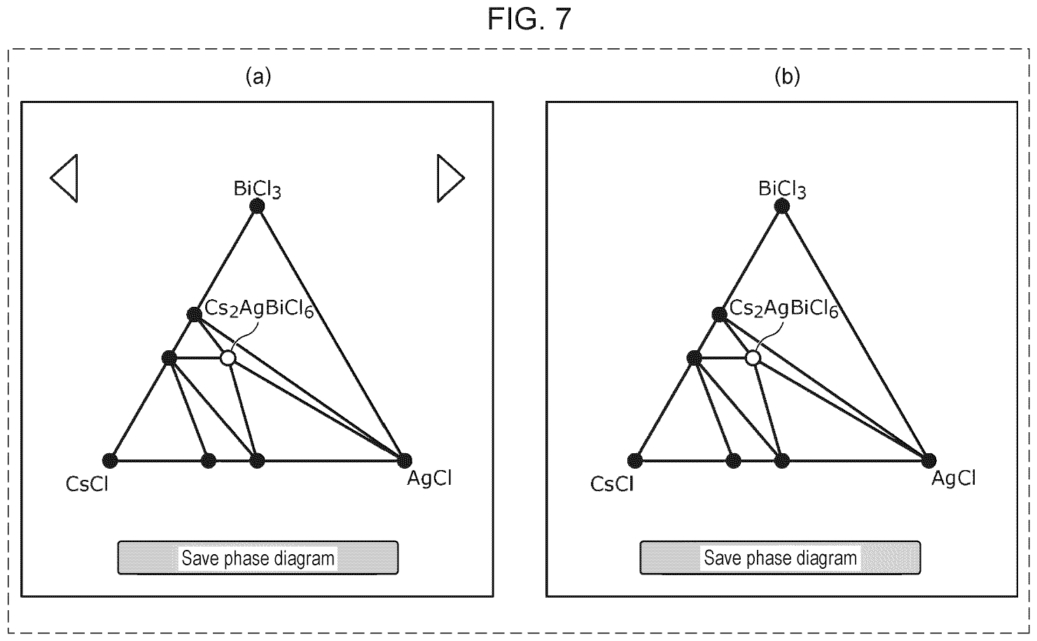

For example, as illustrated in FIG. 7, the second image may include only one phase diagram, instead of the list of the phase diagrams. FIG. 7 is a diagram illustrating examples of the second image displayed in the display 3 in the first embodiment. In the example illustrated in FIG. 7(a), the second image includes any one phase diagram in the multiple phase diagrams generated by the generator 13, a left arrow icon for selecting the previous phase diagram, a right arrow icon for selecting the subsequent phase diagram, and an execution icon “Save phase diagram”. Upon selection of the left arrow icon or the right arrow icon by the user, another phase diagram is displayed in the display 3. In the example illustrated in FIG. 7(b), the second image includes the first phase diagram in sorting of the multiple phase diagrams generated by the generator 13 and an execution icon “Save phase diagram”. In this case, the user cannot select another phase diagram.

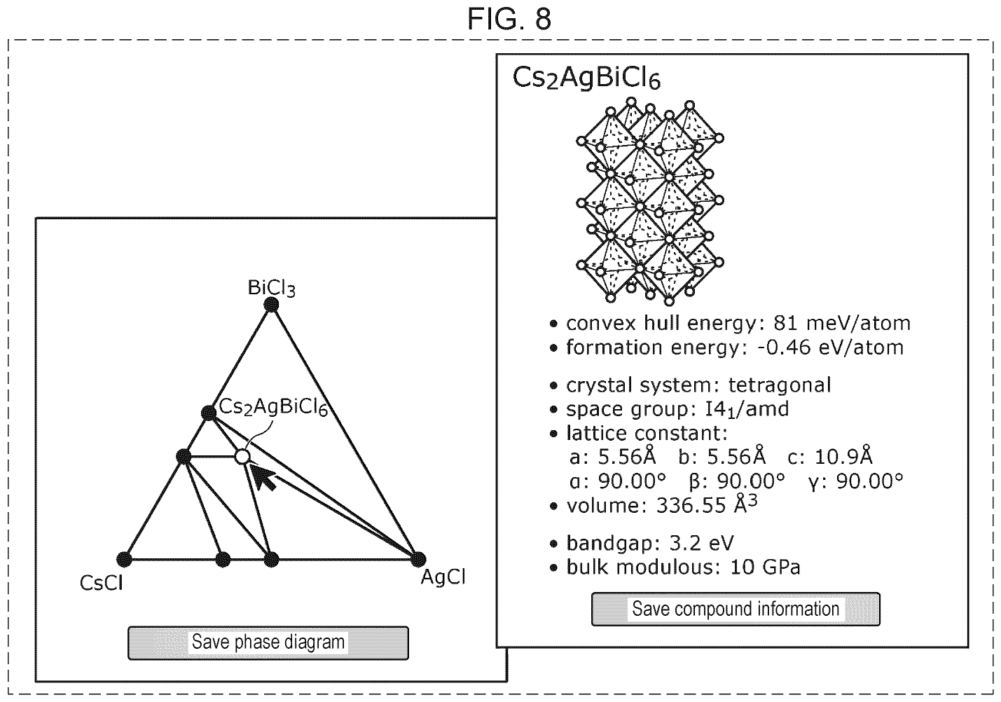

FIG. 8 is a diagram illustrating an example of a third image displayed in the display 3 in the first embodiment. For example, after the user selects any phase diagram on the second image, the third image is displayed in the display 3 with the selected phase diagram. The third image may be displayed in the display 3 by itself. The third image includes compound information concerning the target compound and an execution icon “Save compound information”. The compound information displayed as the third image is, for example, the material property information indicating the material property values, such as the structure information, the energy information, the band gap, the volume, the ionic conductivity, and/or the dielectric constant, of the compound. The structure information may include, for example, the composition of the compound, the crystal system, the space group, the lattice constant, the atomic coordinate, or the like. The energy information may include, for example, the thermodynamic convex hull energy, the formation energy, or the like. Upon selection of the execution icon “Save compound information” on the third image by the user, the compound information concerning the target compound is saved in the second storage 5 in association with the fourth information indicating the phase diagram selected by the user.

### Operation

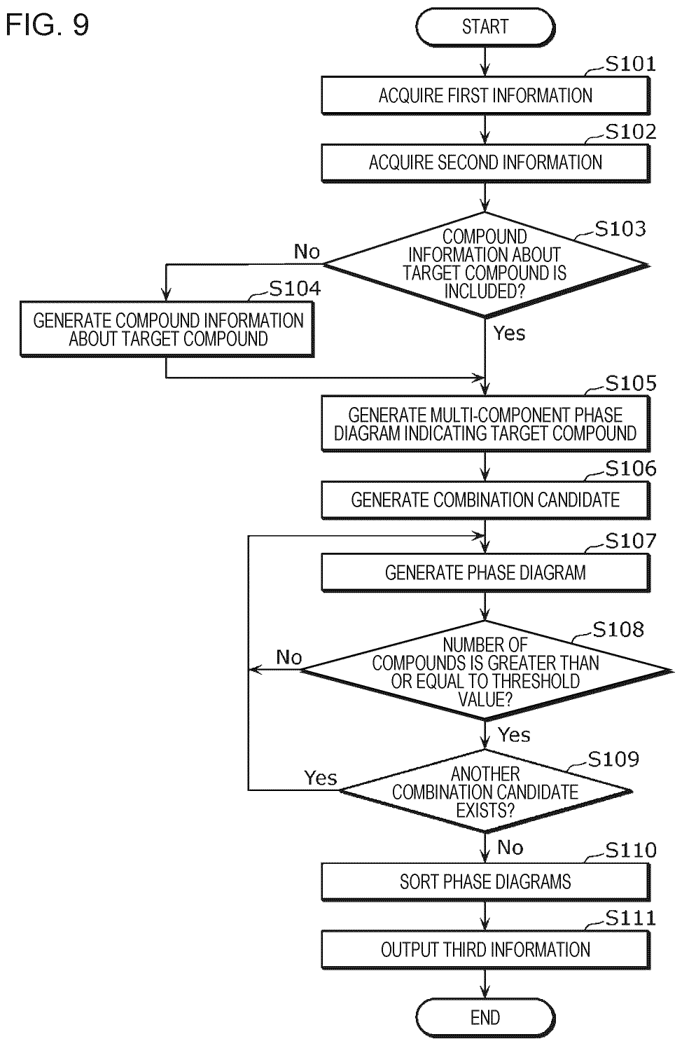

An operation (that is, the information processing method) of the information processing system 100 according to the first embodiment will now be described. FIG. 9 is a flowchart illustrating an example of the operation of the information processing system 100 according to the first embodiment.

### Step S101

The first acquirer 11 acquires the first information. The first information is, for example, the composition information (for example, Cs₂AgBiCl₆) about the target compound, as described above. Upon input (selection) of the first information by the user with the inputter 2 while looking at the first image displayed in the display 3, the first information is acquired by the first acquirer 11, as described above. The first information may be acquired by the first acquirer 11 upon input of original data by the user with the inputter 2 with no reference to the first image.

### Step S102

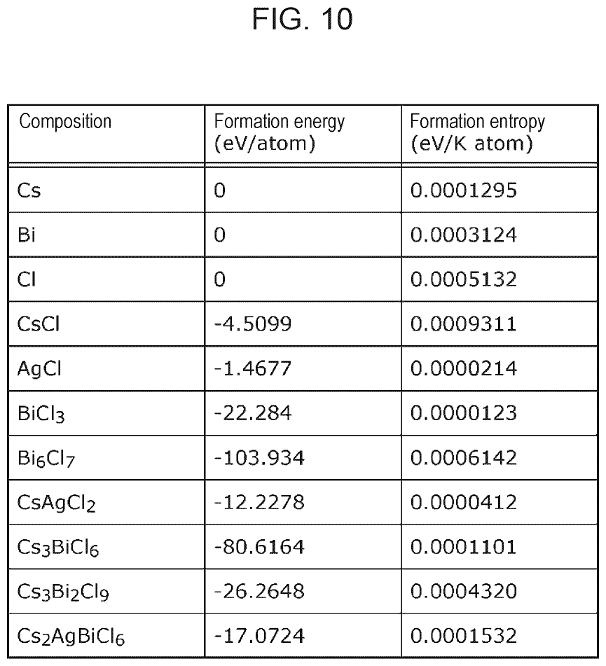

The second acquirer 12 acquires the second information. The second information is information concerning the composition of the target compound, as described above. The second acquirer 12 reads out the data concerning the composition of the target compound, indicated by the first information, from the first storage 4 to acquire the read-out data as the second information. FIG. 10 is a table indicating an example of the second information. The example indicated in FIG. 10 indicates the second information when the target compound is Cs₂AgBiCl₆. As indicated in FIG. 10, the second information includes the composition concerning the target compound and the compound information about the corresponding composition. Here, the formation energy of the corresponding composition and the formation entropy of the corresponding composition correspond to the compound information. The composition is the target compound, the elements (e.g., Cs, Bi, Cl) composing the target compound, or compounds (e.g., CsCl, AgCl, BiCl₃) formed by combining the two or more kinds of elements composing the target compound.

The second information may include all the energy of the corresponding composition, instead of the formation energy of the corresponding composition. Although the compound information about the target compound is included in the second information in the example indicated in FIG. 10, the compound information about the target compound may not be included in the second information.

### Step 103

Referring back to FIG. 9, the generator 13 performs a process to determine whether the compound information about the target compound indicated by the first information is included in the acquired second information based on the first information and the second information that are acquired. If the compound information about the target compound is not included in the second information (No in Step S103), the generator 13 performs Step S104. If the compound information about the target compound is included in the second information (Yes in Step S103), the generator 13 performs Step S105.

### Step S104

The generator 13 performs a process to generate the compound information about the target compound based on the acquired first information. The compound information is, for example, the energy information, such as the formation energy. For example, the generator 13 is capable of predicting the crystal structure based on the composition of the target compound to calculate the energy information about the crystal structure predicted through simulation, such as the first-principles calculation. The generator 13 is capable of calculating (estimating) the energy information about the predicted crystal structure using, for example, a machine learning-based prediction model.

The prediction model is composed of a Graph Neural Network accepting input of a graph structure. The Graph Neural Network is, for example, a Crystal Graph Convolutional Neural Network (CGCNN), a Material Graph Network (MEGNet), or the like. The prediction model is composed of the MEGNet here. The MEGNet is the Graph Neural Network in which not only nodes (nodal points and apexes) and edges (branches and sides) are used as feature quantities but also a global phase quantity representing the feature of the entire target system is further used as a feature quantity.

The prediction model is subjected to the machine learning so as to output the energy information about an arbitrary crystal structure in response to input of the crystal structure using many learning datasets. The learning datasets include the crystal structure as input data and the energy information corresponding to the crystal structure as correct data.

The generator 13 may calculate (estimate) the energy information about the target compound using the prediction model subjected to the machine learning so as to output the energy information about an arbitrary composition in response to input of the composition. After Step S104, the generator 13 performs Step S105.

### Step S105

The generator 13 performs a process to generate (acquire) the multi-component phase diagram indicating the target compound based on the first information and the second information that are acquired. For example, the generator 13 is capable of calculating the thermodynamic convex hull energy from the composition of the compound included in the second information and the formation energy of the composition and searching for a thermodynamically stable compound based on the calculated thermodynamic convex hull energy to generate the multi-component phase diagram indicating the target compound. The thermodynamic convex hull energy is represented by the following formula, for example, when the composition of the target compound is CuInSe₂:

$$Δ {E}_{hull} ( {CuInSe}_{2} ) = max ( Δ {E}_{form} ( {CuInSe}_{2} ) - \frac{Δ {E}_{form} ( {Cu}_{2} Se )}{2} - \frac{Δ {E}_{form} ( {In}_{2} {Se}_{3} )}{2} , 0 )$$

In the above formula, “ΔEₕᵤₗₗ(A) denotes the thermodynamic convex hull energy of a compound “A” and “ΔE_{form}(A)” denotes the formation energy of the compound “A”. If ΔEₕᵤₗₗ(A) is less than zero (0) as in the above formula, then ΔEₕᵤₗₗ(A) is set to zero (0). The formation energy of the target compound can be calculated using the first-principles calculation or the machine learning-based prediction model. The thermodynamic convex hull energy suggests the possibility of synthesis if it is less than or equal to 0.1 eV, as also described in, for example, a paper by WenHao et al. (S. Wenhao, et al, “The thermodynamic scale of inorganic crystalline metastability” Science advances 2.11 (2016): e1600225). In other words, the thermodynamic convex hull energy is an index of the possibility of synthesis of the crystal structure.

### Step S106

The generator 13 performs a process to generate a combination candidate, which is the compound that can be a phase coexisting with the target compound, based on the generated multi-component phase diagram. The combination candidate is CsCl, AgCl, and or like, for example, when the target compound is Cs₂AgBiCl₆.

### Step S107

The generator 13 performs a process to generate the phase diagram based on the multi-component phase diagram and the combination candidate. The phase diagram here is the phase diagram subjected to the dimensionality reduction of the multi-component phase diagram. Specifically, the generator 13 generates a plane or a straight line including the target compound and two combination candidates arbitrarily selected from all the combination candidates as the phase diagram in the multi-component phase diagram.

### Step S108

The generator 13 performs a process to determine whether the number of the compounds included in the generated phase diagram is greater than or equal to a threshold value. The threshold value is, for example, three. The threshold value may be a number greater than three or may be appropriately set by the user. If the number of the compounds included in the generated phase diagram is greater than or equal to the threshold value (Yes in Step S108), the generator 13 performs Step S109. If the number of the compounds included in the generated phase diagram is smaller than the threshold value (No in Step S108), the generator 13 discards the phase diagram and performs Step S107 again. In the second Step S107, the generator 13 excludes the combination candidates that have been selected and arbitrarily selects two combination candidates.

### Step S109

The generator 13 performs a process to determine whether another combination candidate that has not been selected exists. If another combination candidate exists (Yes in Step S109), the generator 13 performs Step S107 again. In the second S107, the generator 13 excludes the combination candidates that have been selected and arbitrarily selects two combination candidates. If another combination candidate does not exist (No in Step S109), the generator 13 performs Step S110.

### Step S110

The generator 13 performs a process to sort the generated one or more phase diagrams. Here, the generator 13 sorts the generated one or more phase diagrams in the descending order of the numbers of the compounds included in the phase diagrams.

### Step S111

The outputter 14 performs a process to output the third information indicating the one or more phase diagrams generated by the generator 13. Here, the outputter 14 performs image generation of the second image representing the third information generated by the generator 13 and displays the second image subjected to the image generation in the display 3 to output the third information.

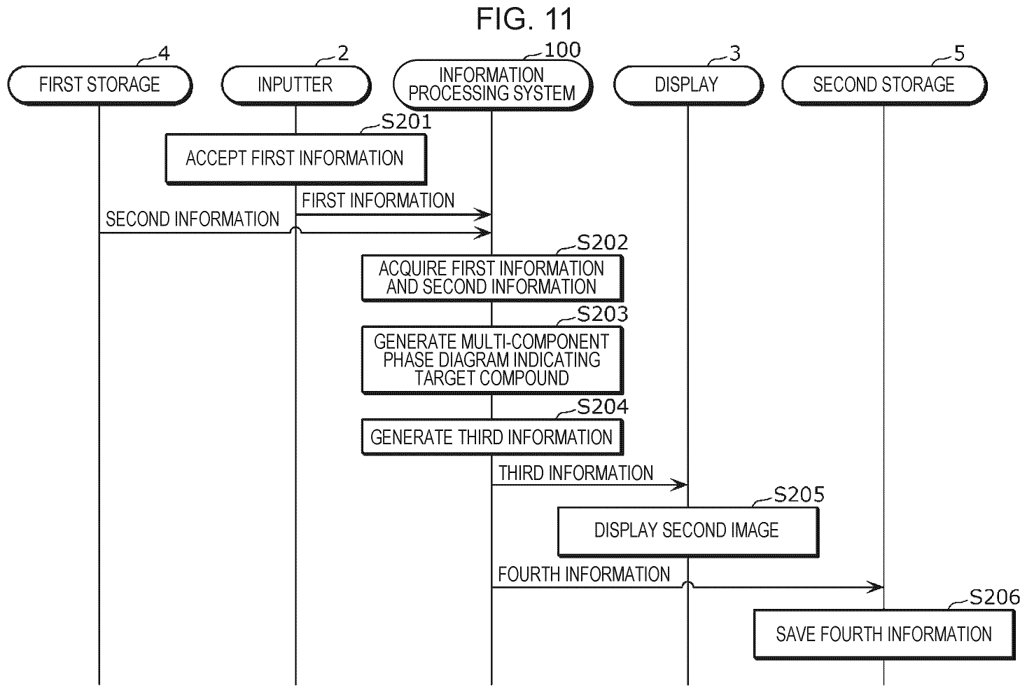

An example of the entire operation including the information processing system 100 according to the first embodiment will now be described with reference to the drawing. FIG. 11 is a sequence diagram illustrating an example of the entire operation including the information processing system 100 according to the first embodiment.

### Step S201

The inputter 2 accepts input of the first information. Here, the user inputs (selects) the first information with the inputter 2 while looking at the first image displayed in the display 3.

### Step S202

The first acquirer 11 in the information processing system 100 acquires the first information accepted with the inputter 2. The second acquirer 12 in the information processing system 100 reads out the data concerning the composition of the target compound, indicated by the first information, from the first storage 4 to acquire the read-out data as the second information.

### Step S203

The generator 13 in the information processing system 100 performs a process to generate (acquire) the multi-component phase diagram indicating the target compound based on the first information and the second information that are acquired. The same processing as in Step S103 and Step S104 (refer to FIG. 9) is performed between Step S202 and Step S203.

### Step S204

The generator 13 in the information processing system 100 generates the third information indicating the one or more phase diagrams. The same processing as in Step S106 to Step S110 (refer to FIG. 9) is performed in Step S204.

### Step S205

The display 3 displays the second image representing the third information output from the outputter 14 in the information processing system 100. The second image is the second image subjected to the image generation.

### Step S206

Upon selection of the phase diagram to be saved by the user while looking at the second image displayed in the display 3, the information processing system 100 supplies the fourth information indicating the selected phase diagram to the second storage 5. The second storage 5 saves the fourth information indicating the phase diagram selected by the user.

As described above, in the first embodiment, it is possible to output the phase diagram which includes the target compound made of the four or more kinds of elements and in which each of the two or three kinds of substances is placed on an apex to be two-dimensionally represented. In other words, in the first embodiment, it is possible to reduce the order of the multi-component phase diagram indicating the target compound so that the phase diagram is two-dimensionally represented so as to include the target compound and the phases coexisting with the target compound or the phases, which are the raw materials. Accordingly, since the phase diagram that is easily visually recognized by users for the multi-component compound is capable of being output in the first embodiment, the user can easily design and develop the target compound, which is the multi-component compound, by referring to the phase diagram.

### Second Embodiment

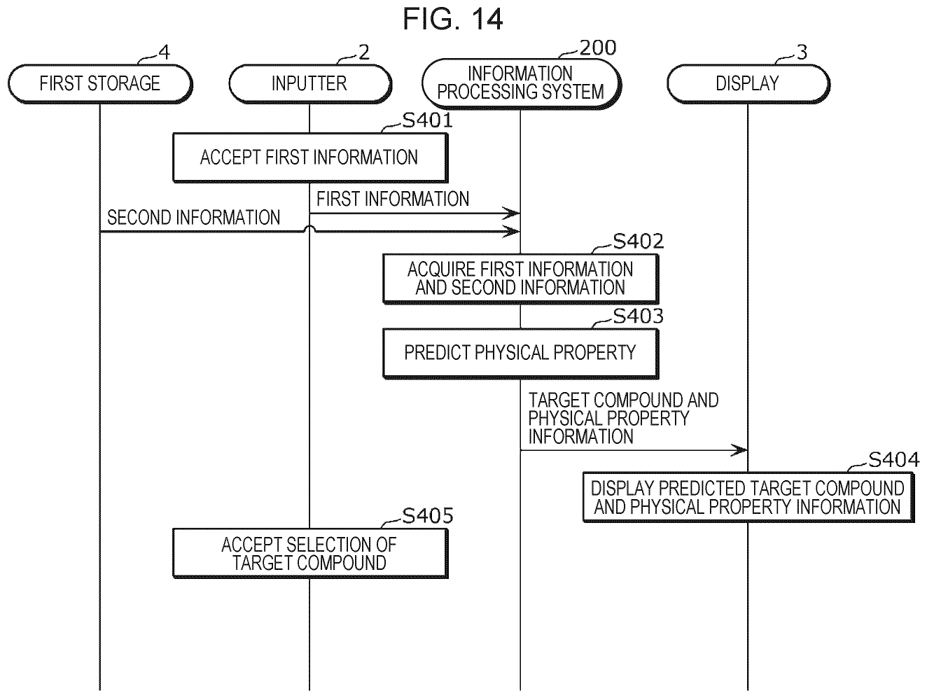

An information processing system 200 (an information processing method or a program) according to a second embodiment of the present disclosure (refer to FIG. 14) will now be described in detail with reference to the drawings. The information processing system 200 according to the second embodiment differs from the information processing system 100 according to the first embodiment in that the first acquirer 11 acquires not the composition information about the target compound but the element information indicating the elements included in the target compound as the first information. Description of the components common to the information processing system 100 according to the first embodiment is omitted herein.

### Examples of Use

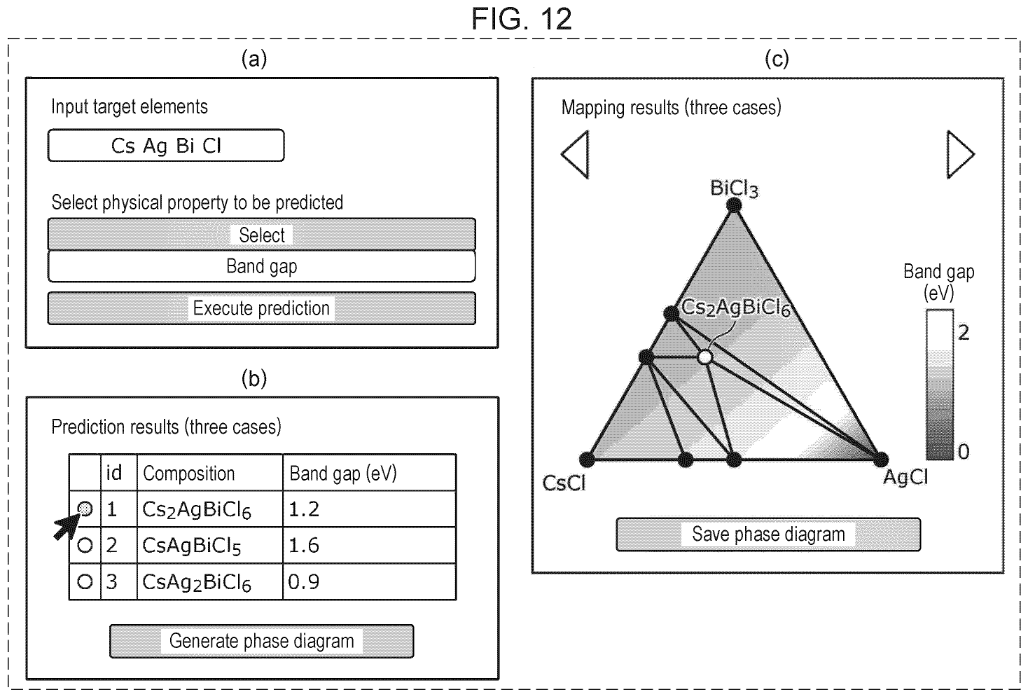

Examples of use of the information processing system 200 according to the second embodiment will now be described. FIG. 12 is a diagram illustrating examples of images displayed in the display 3 in the second embodiment. FIG. 12(a) illustrates an example of the first image displayed in the display 3. In the second embodiment, the first image includes a first information input field used to input the first information, which is the element information (for example, Cs, Ag, Bi, Cl, and the like) about the target compound, a material property selection field used to select a material property to be predicted, and an execution icon “Execute prediction”.

A text box used to specify the elements (“target elements” in FIG. 12(a)) included in the target compound is displayed in the first information input field. The material property of the target compound selectable by the user is displayed in the material property selection field. Although the user selects the band gap as the material property in the example illustrated in FIG. 12(a), another material property may be selectable using, for example, a pull-down menu. The selectable material property is, for example, the band gap, electric conductivity, the ionic conductivity, thermal conductivity, or the dielectric constant.

The user selects the execution icon after inputting desired target elements in the text box and selecting a desired material property. As a result, the first acquirer 11 acquires the element information about the target compound as the first information. In this case, the second acquirer 12 acquires the data about the elements input by the user from the first storage 4 as the second information. The generator 13 predicts one or more target compounds including the target elements based on the first information and the second information to predict the material property value of the predicted one or more target compounds. The fifth information is information indicating the one or more target compounds predicted by the generator 13 and the material property value of the one or more target compounds.

FIG. 12(b) illustrates an example of a fourth image displayed in the display 3. The fourth image is displayed in the display 3 after the user selects the execution icon on the first image and the generator 13 predicts the material property information about the predicted target compounds. The fourth image includes the one or more target compounds predicted by the generator 13, a table indicating a list of the material properties (the band gaps here) of the one or more target compounds predicted by the generator 13, and an execution icon “Generate phase diagram”.

The user selects the execution icon “Generate phase diagram” after selecting the button corresponding to a desired target compound, among the one or more target compounds. The generator 13 generates the one or more phase diagrams for the selected target compound. The outputter 14 outputs the third information indicating the one or more phase diagrams generated by the generator 13. As a result, the second image (refer to FIG. 6(b)) for the target compound selected by the user is displayed in the display 3. Since the second image displayed in the display 3 here is the same as the second image in the first embodiment, description of the second image is omitted herein.

FIG. 12(c) is an example of the second image including an image resulting from enlargement of the phase diagram selected by the user, a left arrow icon for selecting the previous phase diagram, a right arrow icon for selecting the subsequent phase diagram, and an execution icon “Save phase diagram”. In the example illustrated in FIG. 12(c), a heat map in which the magnitude of the material property value (the band gap here) is represented using grayscale is reflected in the phase diagram. The user selects the execution icon “Save phase diagram” on the image. As a result, the fourth information indicating the phase diagram selected by the user is saved in the second storage 5.

### Operation

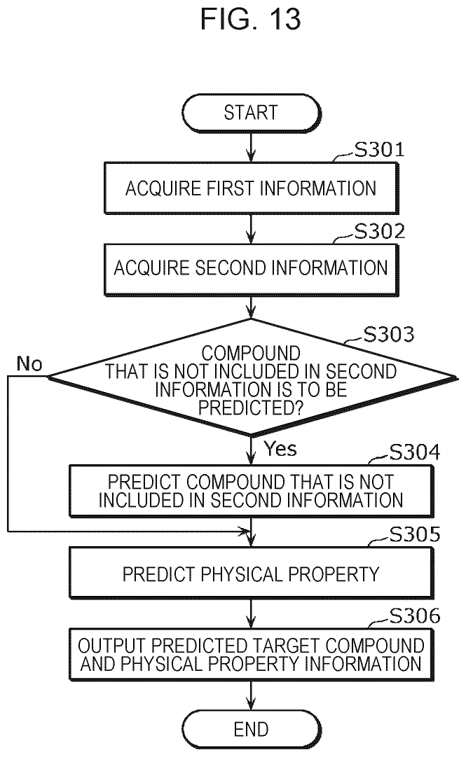

An operation (that is, the information processing method) of the information processing system 200 according to the second embodiment will now be described. FIG. 13 is a flowchart illustrating an example of the operation of the information processing system 200 according to the second embodiment. The processing until the fifth information predicted by the generator 13 is output is described below. Since the processing after the user selects the target compound is the same as that in Steps S101 to S111 (excluding Steps S103 and S104) in the information processing system 100 according to the first embodiment, description of the processing after the user selects the target compound is omitted herein.

### Step S301

The first acquirer acquires the first information. The first information is, for example, the element information (for example, Cs, Ag, Bi, or Cl) about the target compound and the material properties of the target compound, as described above. Upon input (selection) of the first information by the user with the inputter 2 while looking at the first image displayed in the display 3, the first information is acquired by the first acquirer 11, as described above. The first information may be acquired by the first acquirer 11 upon input of original data by the user with the inputter 2 with no reference to the first image.

### Step S302

The second acquirer 12 acquires the second information. The second information is information concerning the composition of the target compound, as described above. The second acquirer 12 reads out the data concerning the composition of the target compound, indicated by the first information, from the first storage 4 to acquire the read-out data as the second information. Here, the second acquirer 12 acquires data about the target elements and data about the compound made of two or more kinds of target elements as the data concerning the composition of the target compound.

### Step S303

The generator 13 performs a process to determine whether the compound that is not included in the acquired second information is to be predicted based on the first information and the second information that are acquired. Whether the compound that is not included in the acquired second information is to be predicted may be set in advance or may be appropriately set by the user. If the compound that is not included in the acquired second information is to be predicted (Yes in Step S303), the generator 13 performs Step S304. If the compound that is not included in the acquired second information is not to be predicted (No in Step S303), the generator 13 performs Step S305.

### Step S304

The generator 13 performs a process to predict the compound that is not included in the second information based on the first information and the second information that are acquired. The generator 13 predicts the compound that is made of all the target elements and that is not included in the second information.

The generator 13 performs the process to predict the one or more target compounds that are made of all the target elements by performing Steps S302 to S304. In other words, the generator 13 predicts the compound made of all the target elements, among the compounds included in the second information, as the target compound. The generator 13 predicts the compound that is not included in the second information and that is made of all the target elements to predict the target compound.

### Step S305

The generator 13 performs a process to predict the material property (for example, the band gap or the like) for each of the predicted one or more target compounds. For example, the generator 13 is capable of predicting the crystal structure based on the composition of the predicted target compound to calculate the material property value of the crystal structure predicted through simulation, such as the first-principles calculation. In addition, the generator 13 is capable of calculating (estimating) the material property value of the predicted crystal structure using, for example, the machine learning-based prediction model. If the material property value of the predicted target compound is included in the second information, the generator 13 may refer to the second information.

### Step S306

The outputter 14 performs a process to output the fifth information predicted by the generator 13. Here, the outputter 14 displays the fourth image representing the fifth information predicted by the generator 13 in the display 3 to output the fifth information.

An example of the entire operation including the information processing system 200 according to the second embodiment will now be described with reference to the drawing. FIG. 14 is a sequence diagram illustrating an example of the entire operation including the information processing system 200 according to the second embodiment. The processing until selection of the target compound by the user is accepted is described below. Since the processing after the user selects the target compound is the same as that in Steps S202 to S206 in the entire operation including the information processing system 100 according to the first embodiment, description of the processing after the user selects the target compound is omitted herein.

### Step S401

The inputter 2 accepts input of the first information. Here, the user inputs (selects) the first information with the inputter 2 while looking at the first image displayed in the display 3.

### Step S402

The first acquirer 11 in the information processing system 200 acquires the first information accepted with the inputter 2. The second acquirer 12 in the information processing system 200 reads out the data concerning the composition of the target compound, indicated by the first information, from the first storage 4 to acquire the read-out data as the second information.

### Step S403

The generator 13 in the information processing system 200 performs a process to predict the material property for each of the predicted one or more target compounds. The same processing as in Step S303 and Step S304 (refer to FIG. 13) is performed between Step S402 and Step S403.

### Step S404

The display 3 displays the fourth image representing the fifth information output from the outputter 14 in the information processing system 200.

### Step S405

The inputter 2 accepts selection of the target compound by the user. Here, the user selects a desired target compound with the inputter 2 while looking at the fourth image displayed in the display 3.

As described above, in the second embodiment, by specifying multiple elements to be included in the target compound even without specification of a desired target compound by the user, it is possible to output the phase diagram that is easily visually recognized by users for the target compound made of the specified multiple elements. Accordingly, in the second embodiment, the user can easily design and develop the target compound, which is the multi-component compound, by referring to the output phase diagram.

### Third Embodiment

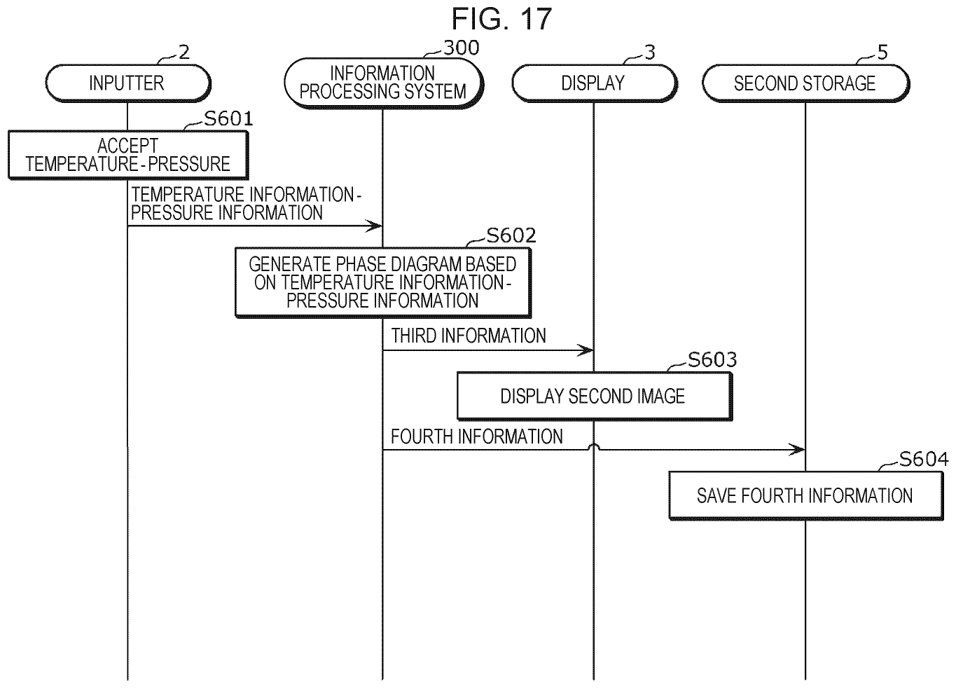

An information processing system 300 (an information processing method or a program) according to a third embodiment of the present disclosure (refer to FIG. 17) will now be described in detail with reference to the drawings. The information processing system 300 according to the third embodiment differs from the information processing system 100 according to the first embodiment in that the outputter 14 outputs the third information indicating the phase diagram considering temperature information and pressure information. Description of the components common to the information processing system 100 according to the first embodiment is omitted herein.

### Examples of Use

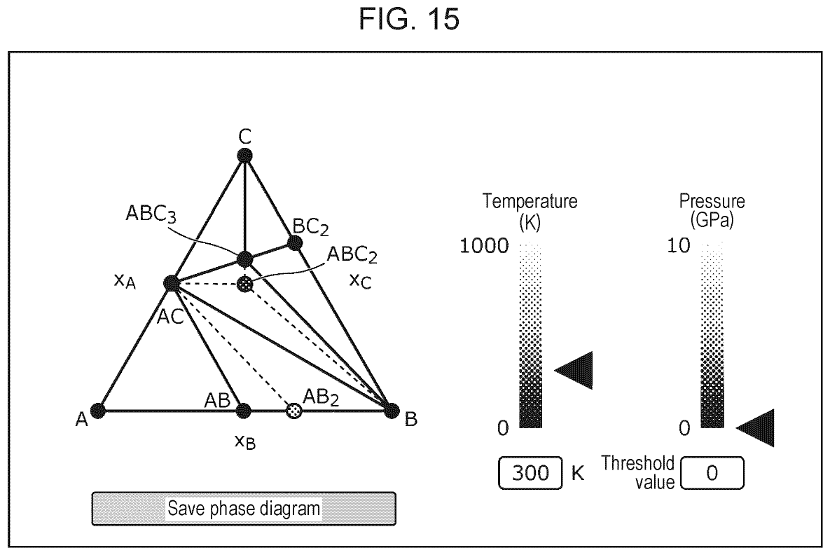

Examples of use of the information processing system 300 according to the third embodiment will now be described. FIG. 15 is a diagram illustrating an example of the second image displayed in the display 3 in the third embodiment. FIG. 15 is an example of the second image including an image resulting from enlargement of the phase diagram selected by the user, a temperature input field used to input the temperature information, a pressure input field used to input the pressure information, and an execution icon “Save phase diagram”.

A bar for specifying a temperature condition of the target compound and a text box for specifying the temperature condition are displayed in the temperature input field. The user may specify the temperature condition using the bar or may specify the temperature condition by inputting the temperature condition in the text box.

A bar for specifying a pressure condition of the target compound and a text box for specifying the pressure condition are displayed in the pressure input field. The user may specify the pressure condition using the bar or may specify the pressure condition by inputting the pressure condition in the text box.

Upon at least one of input to specify the temperature condition in the temperature input field or input to specify the pressure condition in the pressure input field by the user, the generator 13 updates the phase diagram based on the input information (at least one of the temperature information or the pressure information). The outputter 14 outputs the third information indicating the phase diagram updated by the generator 13. As a result, the second image indicating the updated phase diagram is displayed in the display 3. In the example illustrated in FIG. 15, ABC₃, which is the target compound, and ABC₂, which is a thermodynamically stable compound under the specified temperature and pressure environments, are displayed in the phase diagram. A phase thermodynamically coexisting with the compound ABC₂ is displayed by broken lines in the phase diagram. The user selects the execution icon “Save phase diagram” on the image. As a result, the fourth information indicating the phase diagram selected by the user is saved in the second storage 5. For example, predicting a temperature-pressure range in which the target compound can exist in the phase diagram based on the temperature condition and the pressure condition that are input enables the user to design the synthesis conditions of the target compound.

### Operation

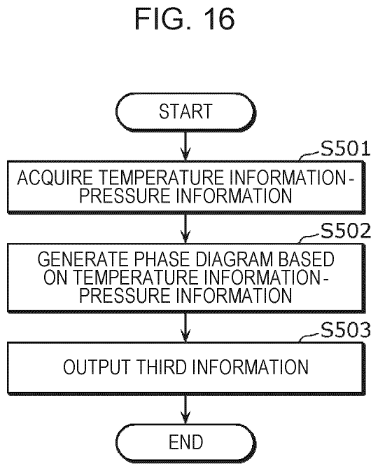

An operation (that is, the information processing method) of the information processing system 300 according to the third embodiment will now be described. FIG. 16 is a flowchart illustrating an example of the operation of the information processing system 300 according to the third embodiment. The processing after the user specifies at least one of the temperature or the pressure is described below. Since the processing before the user specifies at least one of the temperature or the pressure is the same as that in Steps S101 to S111 in the information processing system 100 according to the first embodiment, description of the processing before the user specifies at least one of the temperature or the pressure is omitted herein.

### Step S501

The first acquirer 11 acquires at least one of the temperature information or the pressure information (both the temperature information and the pressure information are acquired in FIG. 16). Upon input of the temperature in the temperature input field by the user with the inputter 2 while looking at the second image displayed in the display 3, the temperature information is acquired by the first acquirer 11. Upon input of the pressure in the pressure input field by the user with the inputter 2 while looking at the second image displayed in the display 3, the pressure information is acquired by the first acquirer 11.

### Step S502

The generator 13 updates the phase diagram selected by the user based on at least one of the acquired temperature information or pressure information (both the temperature information and the pressure information in FIG. 16) to perform a process to generate the phase diagram. For example, the generator 13 calculates the formation energy ΔE_{form} considering the temperature and the pressure based on the formation energy corresponding to the composition of the compound included in the second information, formation enthalpy change ΔH_{form} included in the second information, formation entropy change ΔS_{form} and reaction volume change ΔV_{form}, and at least one of the acquired temperature information or pressure information (both the temperature information and the pressure information here). The formation energy ΔE_{form} is represented by the following formula:

$$Δ {E}_{form} = Δ {H}_{form} - T Δ {S}_{form} + P Δ {V}_{form}$$

In the above formula “T” denotes temperature and “P” denotes pressure. The generator 13 updates the phase diagram selected by the user based on the calculated formation energy to generate the phase diagram. The temperature range is, for example, 0 to 2,000 K. The pressure range is, for example, 0 to 1 MPa. The temperature range is not limited to the above one. The same applies to the pressure range.

### Step S503

The outputter 14 performs a process to output the third information indicating the phase diagram generated by the generator 13. Here, the outputter 14 displays the second image representing the third information generated by the generator 13 in the display 3 to output the third information.

An example of the entire operation including the information processing system 300 according to the third embodiment will now be described with reference to the drawing. FIG. 17 is a sequence diagram illustrating an example of the entire operation including the information processing system 300 according to the third embodiment. The processing after the user specifies at least one of the temperature or the pressure is described below. Since the processing before the user specifies at least one of the temperature or the pressure is the same as that in Steps S201 to S205 in the entire operation including the information processing system 100 according to the first embodiment, description of the processing before the user specifies at least one of the temperature or the pressure is omitted herein.

### Step S601

The inputter 2 accepts input of at least one of the temperature or the pressure (both the temperature and the pressure here). Here, the user inputs at least one of the temperature or the pressure with the inputter 2 while looking at the second image displayed in the display 3.

### Step S602

The generator 13 in the information processing system 300 updates the phase diagram selected by the user based on at least one of the acquired temperature information or pressure information (both the temperature information and the pressure information here) to perform a process to generate the phase diagram.

### Step S603

The display 3 displays the second image representing the third information output from the outputter 14 in the information processing system 300.

### Step S604

Upon selection of the phase diagram to be saved while looking at the second image displayed in the display 3 by the user, the information processing system 300 supplies the fourth information indicating the selected phase diagram to the second storage 5. The second storage 5 saves the fourth information indicating the phase diagram selected by the user.

As described above, in the third embodiment, it is possible to output the phase diagram that is easily visually recognized by users for the target compound in consideration of at least one of the temperature or the pressure. Accordingly, in the third embodiment, the user can easily design and develop the target compound, which is the multi-component compound, in consideration of at least one of the temperature or the pressure, by referring to the output phase diagram.

### Fourth Embodiment

An information processing system (an information processing method or a program) according to a fourth embodiment of the present disclosure will now be described in detail with reference to the drawing. The information processing system according to the fourth embodiment differs from the information processing system 100 according to the first embodiment in that the outputter 14 outputs the third information indicating the phase diagram having energy axes added thereto. Description of the components common to the information processing system 100 according to the first embodiment is omitted herein.

### Examples of Use

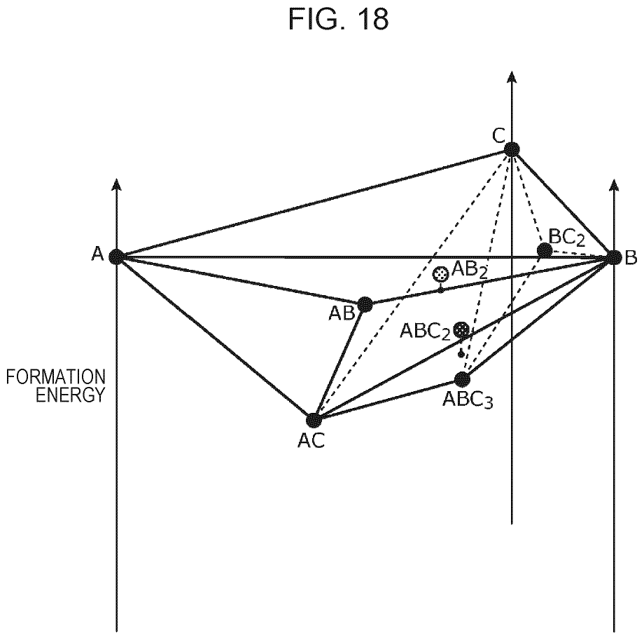

Examples of use of the information processing system according to the fourth embodiment will now be described. FIG. 18 is a diagram illustrating an example of the second image displayed in the display 3 in the fourth embodiment. FIG. 18 is an example of the second image including an image resulting from enlargement of the phase diagram selected by the user. Illustration of an execution icon “Save phase diagram” is omitted in the example in FIG. 18.

As illustrated in FIG. 18, one or more energy axes (an energy axis passing through an element A, an energy axis passing through an element B, and an energy axis passing through an element C here) are added to the phase diagram. Each energy axis represents the magnitude of the formation energy. On the respective energy axes, the formation energies of the element A, the element B, and the element C are zero. The magnitude of the energy represented by each energy axis is not limited to the magnitude of the formation energy and may be the magnitude of another energy. When the one or more energy axes are added to a two-dimensional plane, the phase diagram represented on the two-dimensional plane may be converted into a three-dimensional model to be displayed in the display 3.

In the information processing system according to the fourth embodiment, the outputter 14 outputs the third information that indicates the phase diagram and that further includes information indicating the energy of each substance (element or compound) included in the phase diagram. Accordingly, the display controller 30 is capable of displaying the phase diagram having the one or more energy axes added thereto in the display 3.

As described above, in the fourth embodiment, it is possible to output the phase diagram having the one or more energy axes added thereto. Accordingly, in the fourth embodiment, the stability of the compounds included in the phase diagram is easily visually recognized. For example, although the user can understand whether the compounds included in the phase diagram are thermodynamically stable in the first embodiment, it is difficult for the user to understand how much the compounds are stable. In contrast, in the fourth embodiment, the user can easily visually understand how much each compound is stable or how much the target compound is unstable by referring to the phase diagram having the one or more energy axes added thereto.

### Modifications

Although the information processing system (the information processing method) according to one or more aspects of the present disclosure is described above based on the respective embodiments, the present disclosure is not limited to the embodiments. Various modifications obvious to a person skilled in the art may be made to the respective embodiments without departing from the spirit and scope of the present disclosure. Modes resulting from combination of components in the multiple different embodiments may be included in the present disclosure.

For example, in the respective embodiments described above, the second image displayed in the display 3 may further include the multi-component phase diagram, in addition to the phase diagram, as in the example illustrated in FIG. 3. In other words, the display controller 30 may display the second image further including the multi-component phase diagram in the display 3.

For example, the plane that is cut out may be hatched in the multi-component phase diagram displayed in the display 3, as in the example illustrated in FIG. 3. In other words, the display controller 30 may display the second image in which a plane or a line (a hatched portion) representing at least one phase diagram is superimposed on the multi-component phase diagram in the display 3.

Although the first storage 4 and the second storage 5 are realized by different recording media in each embodiment described above, the first storage 4 and the second storage 5 are not limited to this. For example, the first storage 4 and the second storage 5 may be realized by the same recording medium.

Although the first acquirer 11 and the second acquirer 12 are different acquirers in each embodiment described above, the first acquirer 11 and the second acquirer 12 may be realized by the same acquirer.

Although the information processing system is composed of the first acquirer 11, the second acquirer 12, the generator 13, and the outputter 14 in each embodiment described above, the information processing system is not limited to this. For example, the information processing system may be composed of the display controller 30 and the display 3 in each embodiment described above, as illustrated by “100A” in FIG. 5.

In the embodiments described above, each component may be composed of dedicated hardware or may be realized by executing a software program appropriate for each component. Each component may be realized by a program executer, such as a central processing unit (CPU) or a processor, that reads out a software program recorded on a recording medium, such as a hard disk or a semiconductor memory, and executes the read-out software program.

The following cases are also included in the present disclosure.
(1) At least one apparatus described above is specifically a computer system composed of a microprocessor, a ROM, a RAM, a hard disk unit, a display, a keyboard, a mouse, and so on. A computer program is stored in the RAM or the hard disk unit. The microprocessor that works in accordance with the computer program causes the at least one apparatus to achieve its function. The computer program is composed by combining multiple pieces of instruction code indicating instructions to the computer in order to achieve a certain function.
(2) Part or all of the components composing the at least one apparatus may be composed of one system large scale integration (LSI). The system LSI is a super multifunctional LSI manufactured by integrating multiple components on one chip and, specifically, is a computer system including a microprocessor, a ROM, a RAM, and so on. A computer program is stored in the RAM. The microprocessor that works in accordance with the computer program causes the system LSI to achieve its function.
(3) Part or all of the components composing the at least one apparatus may be composed of an integrated circuit (IC) card removable from the apparatus or a single module. The IC card or the module is a computer system including a microprocessor, a ROM, a RAM, and so on. The IC card or the module may include the super multifunctional LSI. The microprocessor that works in accordance with the computer program causes the IC card or the module to achieve its function. The IC card or the module may have tamper resistance.
(4) The present disclosure may be realized by the methods described above. The present disclosure may be realized by a computer program realizing the above methods, which is executed by a computer, or may be realized by digital signals composed of the computer program.

The present disclosure may be realized by recording the computer program or the digital signals on a computer-readable recording medium, such as a flexible disk, a hard disk, a compact-disc (CD)-ROM, a digital versatile disc (DVD), a DVD-ROM, a DVD-RAM, a Blu-ray (registered trademark) disc (BD), or a semiconductor memory. The present disclosure may be realized by the digital signals recorded on the recording medium.

The present disclosure may be realized by transmitting the computer program or the digital signals via an electric telecommunication line, a wireless or wired communication line, a network typified by the Internet, data broadcasting, or the like.

The present disclosure may be realized by recording the program or the digital signals on the recording medium to transfer the program or the digital signals to another independent computer system or transferring the program or the digital signals to another independent computer system via a network or the like.

The present disclosure has an effect of, for example, appropriately supporting search for synthesis process of inorganic materials by the user and is usable for a computer apparatus or a system for displaying information concerning the generating process.

What is claimed is:

1. An information processing method performed by a computer, the method comprising:
acquiring a multi-component phase diagram which includes a target compound made of four or more kinds of elements and in which each of the four or more kinds of elements is placed on an apex to be at least three-dimensionally represented;
extracting one or more phase diagrams in which each of two or three kinds of substances is placed on an apex to be two-dimensionally represented from the acquired multi-component phase diagram; and
outputting the acquired phase diagram,
wherein at least one of the two or three kinds of substances is a compound made of at least two kinds of elements, among the four or more kinds of elements.

2. The information processing method according to claim 1,
wherein the multi-component phase diagram includes multiple compounds formed by combining two or more kinds of elements, among the four or more kinds of elements, and
wherein, in the extracting the phase diagram, the phase diagram in which at least one compound in the multiple compounds is placed at a position other than the apex is extracted.

3. The information processing method according to claim 1,
wherein, in the outputting the phase diagram, the phase diagram is one or more different phase diagrams and an image indicating the one or more phase diagrams is displayed in a display.

4. The information processing method according to claim 3,
wherein, in the outputting the phase diagram, the one or more phase diagrams are arranged in a descending order of numbers of the compounds included in the phase diagrams.

5. The information processing method according to claim 1,
wherein, in the outputting the phase diagram, the image in which a plane or a line representing at least one phase diagram is superimposed on the multi-component phase diagram is displayed in the display.

6. The information processing method according to claim 1,
wherein the one or more phase diagrams are each represented in a triangle and are differentiated in at least one of a shape of the triangle, a substance corresponding to each apex of the triangle, or an area of the triangle.

7. The information processing method according to claim 1,
wherein, in the outputting the phase diagram, the image representing the phase diagram generated based on at least one of a temperature condition or a pressure condition of the target compound is displayed in the display.

8. The information processing method according to claim 1,
wherein, in the outputting the phase diagram, the image representing the phase diagram that further includes an energy axis indicating energy of each of multiple substances included in the phase diagram is displayed in the display.

9. A recording medium provided with a program to cause a computer to perform processing, the recording medium being a non-transitory computer-readable recording medium, the processing comprising:
acquiring a multi-component phase diagram which includes a target compound made of four or more kinds of elements and in which each of the four or more kinds of elements is placed on an apex to be at least three-dimensionally represented;
extracting one or more phase diagrams in which each of two or three kinds of substances is placed on an apex to be two-dimensionally represented from the acquired multi-component phase diagram; and
outputting the acquired phase diagram,
wherein at least one of the two or three kinds of substances is a compound made of at least two kinds of elements, among the four or more kinds of elements.

10. An information processing system comprising:
a generator that acquires a multi-component phase diagram which includes a target compound made of four or more kinds of elements and in which each of the four or more kinds of elements is placed on an apex to be at least three-dimensionally represented and that extracts one or more phase diagrams in which each of two or three kinds of substances is placed on an apex to be two-dimensionally represented from the acquired multi-component phase diagram; and
an outputter that outputs the acquired phase diagram,
wherein at least one of the two or three kinds of substances is a compound made of at least two kinds of elements, among the four or more kinds of elements.
